(19) United States
(12) Patent Application Publication
(10) Pub. No.: US 2025/0382294 A1
(43) Pub. Date: Dec. 18, 2025

(54) NOVEL VAPOCHROMIC ORGANIC MATERIAL FOR DETECTION OF VOLATILE ORGANIC COMPOUND

(71) Applicant: Korea University Research and Business Foundation, Sejong Campus, Sejong (KR)

(72) Inventor: Kyung-Ryang WEE, Sejong (KR)

(73) Assignee: Korea University Research and Business Foundation, Sejong Campus, Sejong (KR)

(21) Appl. No.: 19/280,733

(22) Filed: Jul. 25, 2025

(30) Foreign Application Priority Data
Apr. 19, 2024 (KR) 10-2024-0052601
Jun. 3, 2024 (KR) 10-2024-0072430

(51) Int. Cl. C07D 471/04; C09K 11/06

(52) U.S. Cl. CPC C07D 471/04; C09K 11/06

(57) ABSTRACT

Is disclosed a Donor-Acceptor-Donor (D-A-D) positional isomer-based organic material compound containing naphthalene diimide (NDI).

### CROSS-REFERENCE TO RELATED APPLICATION

The application claims priority under 35 U.S.C. § 119 to the Korean Patent Application No. 10-2024-0052601 filed on Apr. 19, 2024 and the Korean Patent Application No. 10-2024-0072430 filed on Jun. 3, 2024, all of which are incorporated by reference herein.

### TECHNICAL FIELD

The description below relates to a novel vapochromic organic material, and more specifically, to a novel vapochromic organic material which is based on a naphthalene diimide (NDI) acceptor in a Donor-Acceptor-Donor (D-A-D) system and is able to selectively induce vaporchromic properties by controlling a porous molecule arrangement mode through a positional isomeric effect arising from the substitution position of donor molecules.

### BACKGROUND OF THE DISCLOSURE

Volatile Organic Compounds (VOCs) are molecules with a low boiling point and are liquid and gaseous organic compounds that are highly volatile and easily evaporate into the atmosphere. VOCs, which are commonly generated in manufacturing sites and households, are harmful to human health, most of them are easily absorbed into the body through respiration and skin contact due to their low molecular weight, and continuous exposure and short-term exposure to high concentrations cause cancer in major organs, asphyxiation due to respiratory distress, suppression of on the central nervous system, etc. Typically, aromatic hydrocarbons such as benzene, toluene, ethylbenzene, and xylene isomer (BTEX), which are carcinogens, and formaldehydes that cause sick building syndrome, are included in regulated VOCs. Therefore, it is essential to rapidly detect such VOCs on-site in order to identify an air pollution level and prevent VOC exposure and/or intoxication-related accidents and environmental contamination in advance.

However, it is very difficult to detect VOCs in real time in a local environment. Previously, samples had to be repeatedly taken and transported to laboratories that had high-performance liquid chromatography and gas chromatography. To overcome this, VOC detection sensors based on electrochemical, semiconductor, and photoionization methods have been developed, they still face challenges such as high operating power, high cost, and limitations in target selectivity and sensitivity. Therefore, it is necessary to research and develop sensor materials that enable on-site detection of VOCs while retaining the advantages of existing VOC detection sensors.

### SUMMARY

A Donor-Acceptor-Donor (D-A-D) positional isomer-based organic material compound containing naphthalene diimide (NDI) is provided.

In addition, vaporchromism refers to a phenomenon in which changes in color and/or luminescence color may be exhibited in response to specific gases and vapors, and it may be applied to provide vaporchromic molecular materials which can be utilized in the development of photochemical sensors for the simple on-site detection of volatile organic compounds (VOCs).

A Donor-Acceptor-Donor (D-A-D) positional isomer-based organic material compound containing naphthalene diimide (NDI) is provided.

According to an aspect, the donor-acceptor-donor positional isomer may be utilized to manipulate a single molecular structure to control an organic porous molecular arrangement.

According to another aspect, vaporchromic properties for Volatile Organic Compounds (VOCs) may be selectively induced by controlling the organic porous molecular arrangement.

According to another aspect, at least one of color and luminescence color of the organic material compound may be changed in response to a particular gas or vapor.

According to another aspect, the organic material compound may be expressed by the following formula 1.

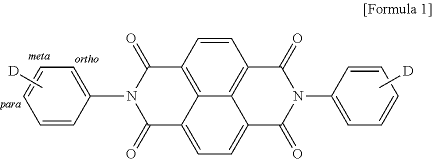

According to another aspect, a phenyl group may be introduced between the napthalendiamide and a donor unit, and the donor unit in the donor-acceptor-donor positional isomer may be substituted at one of positions of ortho-, meta-, and para- of the phenyl.

According to another aspect, the shape of a molecular building block of the donor-acceptor-donor positional isomer may be controlled to be one of Z-shaped, quasi-Z-shaped, or linear as the donor unit is substituted at one of the positions of ortho-, meta-, and para- of the phenyl.

According to another aspect, a pore volume may decrease in the order of Z-shaped, quasi-Z-shaped, and linear according to the shape of the molecular building block.

According to another aspect, the donor unit in the donor-acceptor-donor positional isomer may include triphenylamine (TPA).

According to another aspect, a donor unit in the donor-acceptor-donor positional isomer may be expressed by the following formula 2.

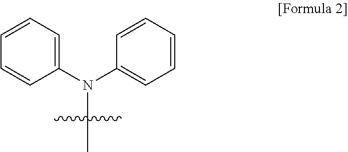 [SMILES: *N(c1ccccc1)c1ccccc1]

According to another aspect, a donor unit in the donor-acceptor-donor positional isomer may be expressed by the following formula 3.

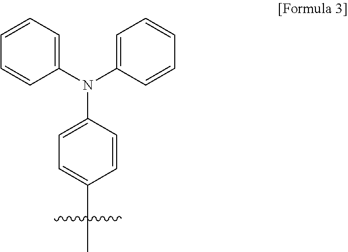 [SMILES: *c1ccc(N(c2ccccc2)c2ccccc2)cc1]

According to another aspect, a donor unit in the donor-acceptor-donor positional isomer may be expressed by the following formula 4.

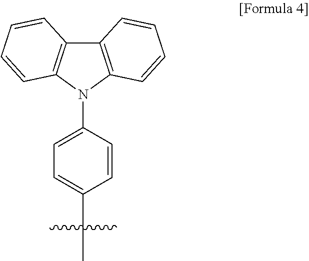 [SMILES: *c1ccc(-n2c3ccccc3c3ccccc32)cc1]

According to another aspect, a donor unit in the donor-acceptor-donor positional isomer may be expressed by the following formula 5.

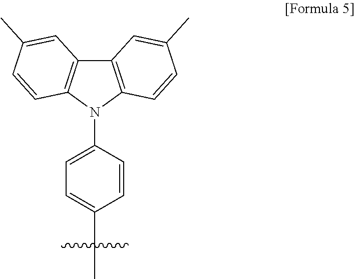

According to another aspect, a donor unit in the donor-acceptor-donor positional isomer may be expressed by the following formula 6.

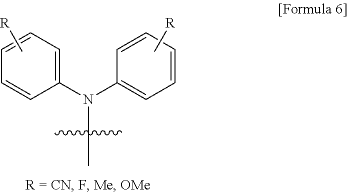

According to another aspect, a donor unit in the donor-acceptor-donor positional isomer may be expressed by the following formula 7.

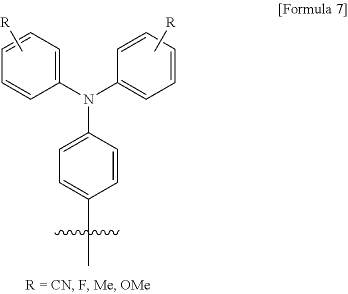

According to another aspect, a donor unit in the donor-acceptor-donor positional isomer may be expressed by the following formula 8.

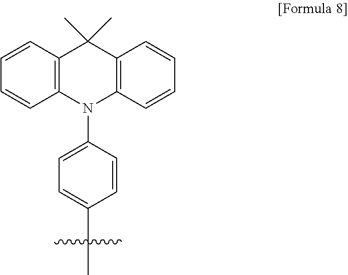 [SMILES: *c1ccc(N2c3ccccc3C(C)(C)c3ccccc32)cc1]

According to another aspect, a donor unit in the donor-acceptor-donor positional isomer may be expressed by the following formula 9.

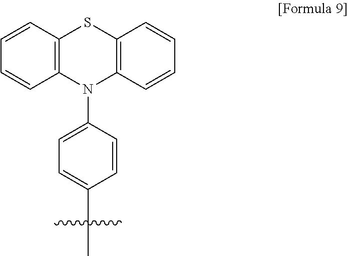 [SMILES: *c1ccc(N2c3ccccc3Sc3ccccc32)cc1]

According to another aspect, a donor unit in the donor-acceptor-donor positional isomer may be expressed by the following formula 10.

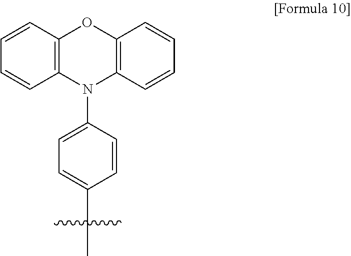 [SMILES: *c1ccc(N2c3ccccc3Oc3ccccc32)cc1]

### BRIEF DESCRIPTION OF THE DRAWINGS

FIG. 1 is a view showing an example of a positional isomer dependent molecular building block (NDI-TO, NDI-TM, and NDI-TP) according to an embodiment of the disclosure.

FIG. 2 is a view showing the stacking structures of (a) NDI-TO, (b) NDI-TM, and (c) NDI-TP, and the normalized distances (dnorm) mapped onto the Hirshfeld surfaces of (d) NDI-TO, (e) NDI-TM, and (f) NDI-TP according to an embodiment of the disclosure.

FIG. 3 is a view showing (g) a bar chart of main non-covalent interactions % obtained from a two-dimensional (2D) fingerprint plot for all compounds, (h) a 2D fingerprint plot generated from dnorm for all interactions and O—H/H—O interactions, and (i) an example of void surfaces of NDI-TO, NDI-TM, and NDI-TP according to an embodiment of the disclosure.

FIG. 4 is view showing examples of a main void and void surfaces of four or more unit cells for (a) NDI-TO, (b) NDI-TM, and (c) NDI-TP according to an embodiment of the disclosure.

FIG. 5 is a view showing examples of low and high magnification Scanning Electron Microscopy (SEM) images for (d-f) NDI-TO, (g-i) NDI-TM and (j-l) NDI-TP, and examples of packing modes for (m) NDI-TO, (n) NDI-TM, and (o) NDI-TP according to an embodiment of the disclosure.

FIG. 6 shows examples of emission spectra of NDI-TO, NDI-TM, and NDI-TP when exposed to various vapors according to an embodiment of the disclosure.

FIG. 7 is a view showing examples of images of NDI-TO (top), NDI-TM (middle), and NDI-TP (bottom) powder in its original state exposed to various vapors under a 365 nm UV lamp according to an embodiment of the disclosure.

FIG. 8 is a view showing examples of time-resolved photoluminescent decays of (c) NDI-TO, (d) NDI-TM and (e) NDI-TP extracted from the relevant fluorescence lifetime images, and examples of fluorescence lifetime images of (f) NDI-TO, (g) NDI-TM and (h) NDI-TP when in contact with acetonitrile (ACN) vapor according to an embodiment of the disclosure.

FIG. 9 is a view showing examples of images of NDI-TP (top), NDI-TM (middle), and NDI-TO (bottom) powder after exposure to various vapors under ambient light for 24 hours according to an embodiment of the disclosure.

FIG. 10 is a view showing an example of changes in color of a rose drawn with NDI-TP using a mask on filter paper under ambient light when sequentially exposed to o-xylene, dichloromethane (DCM), toluene (Tol), acetone (Ace), and tetrahydrofuran (THF) vapors for 24 hours according to an embodiment of the disclosure.

FIG. 11 is a view showing an example of an original state of NDI-TP and experimental Powder X-Ray Diffraction (PXRD) patterns of NDI-TP powder after exposure to Tol, cyclohexane (CyHx) and para-xylene vapors, along with a simulation pattern of the single crystal structure of NDI-TP according to an embodiment of the disclosure.

FIG. 12 is a view showing examples of thermogravimetric analysis (TGA) curves of NDI-TO, NDI-TM, and NDI-TP powder samples after exposure to Tol vapor according to an embodiment of the disclosure.

FIG. 13 is a view showing an example of the correlation between a single molecular shape-dependent molecular arrangement and vapochromism behavior according to an embodiment of the disclosure.

### DETAILED DESCRIPTION

The present disclosure may be subject to various modifications, and have various embodiments. Therefore, specific embodiments will be described in detail below with reference to the accompanying drawings.

In describing the disclosure, the detailed descriptions of the related art may be omitted if deemed to obscure the gist of the disclosure.

Over the past few decades, strategies such as the formation of host-guest charge-moving complexes and the introduction of ring-cage compounds have been used to design vapochromic materials. However, in order to increase the applicability of vapochromic materials, there is still a high demand for an effective design strategy that can control the photophysical properties and bring various functions and performance improvements. Vapochromism is determined by the molecular arrangement and interactions between molecules, which are highly diverse and intricate, and other interactions occur in contact with Volatile Organic compounds (VOCs), making it difficult to predict the vaporchromic properties. Therefore, it is necessary to establish an effective strategy for achieving high-performance vapochromism materials by identifying the correlation between the arrangement and structural properties of molecular materials and vapochromism.

Among the porous materials, the flexible Metal-Organic Frameworks (MOFs) and Covalent Organic Frameworks (COFs) have received considerable research interest in materials science, as they can undergo structural changes in the solid state in response to external stimuli such as pressure, light, temperature, and guest molecules. Their flexibility may be usually obtained through the introduction of structurally changeable organic ligand. This allows greater degrees of freedom in the molecular arrangement, allowing structural changes to be made in response to external stimuli, which can result in significant discoloration and changes in emission characteristics.

Based on this fact, a porous molecular arrangement consisting of pure organic molecules is expected to have greater flexibility since there is no strong covalent and coordination bonds contained in MOFs and COFs, leading to improved vapochromic performance and various functions. Based on the above, embodiments of the disclosure use organic molecules to implement and control porous molecular arrangements with maximum flexibility to provide high-performance vapochromic materials.

Despite the advantages of using organic molecules, the study of the arrangement of organic porous molecules formed by noncovalent bonds is still limited to flexible MOFs and COFs. Therefore, using organic molecules to form a regular porous molecular arrangement is challenging and difficult. For example, organic molecular-based molecular arrangements consist of noncovalent interactions such as van der Waals and hydrogen bonds, and interactions between these molecules are generally weaker and less directional than covalent and coordination bonds. Forming and controlling porous molecular arrangements is a challenging task, especially since organic molecules tend to form dense arrangements with minimal pore volumes to maximize interaction with adjacent molecules.

Thus, to overcome this general tendency, a positional isomer can be used in a naphthalene diimide (NDI)-based Donor-Acceptor-Donor (D-A-D) system that induces directional intermolecular interactions to manipulate the shape of a single-molecule building block. Positional isomers represent different molecular arrangements due to differences in a structural form, and can induce changes in the photophysical properties of material molecules. In particular, using positional isomers may allow the monolecular structure to be gradually manipulated into a structure that cannot be densely filled when forming a molecular arrangement, thereby forming and controlling an organic porous molecular arrangement. Ultimately, changes in vapochromism by controlled organic porous molecular arrangements can provide insight into the design of high-performance vapochromic materials.

As such, embodiments of the disclosure may provide new vaporchromic organic materials that can selectively induce vaporchromic properties by modulating porous molecular arrangements through positional isomer effects for effective VOCs detection.

Here, to construct a self-assembled molecular building block that can efficiently control porous molecular arrangements, a positional isomer vaporchromic organic material based on the D-A-D system may be represented by the following formula 1.

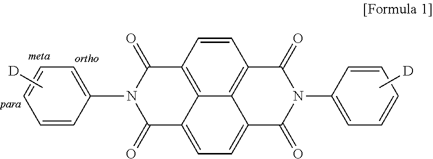

NDI-based D-A-D molecular skeletons can support self-assembly through noncovalent interactions promoted by inducing intermolecular donor-acceptor and intermolecular hydrogen bonds. Here, NDI is an acceptor, D represents a donor unit, and D may be substituted among ortho-, meta-, and para-positions of the phenyl. In addition, D may be expressed by one of the following formulas 2 to 10.

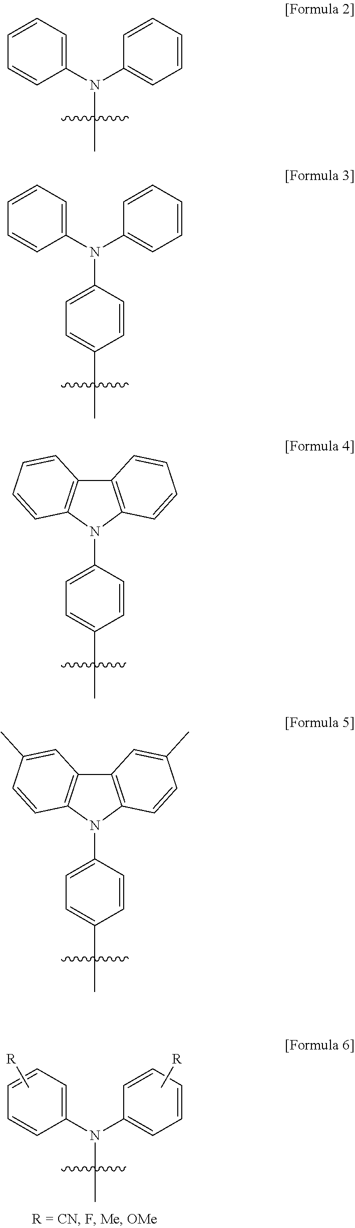

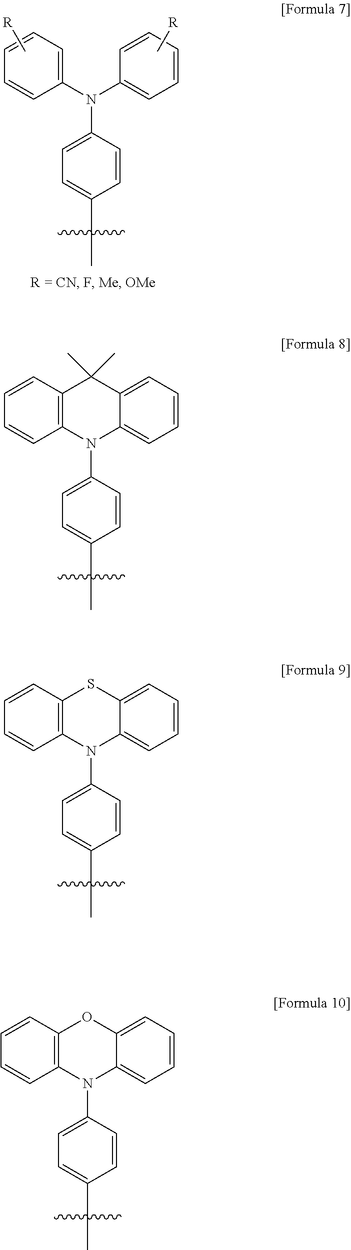

As such, embodiments of the disclosure may provide a positional isomer vaporchromic organic material based on the D-A-D system. A phenyl group is introduced between the NDI and the donor, and the shape of the D-A-D molecular building block may be controlled by Z-shaped, quasi-Z-shaped, and linear through ortho-, meta-, and para-substitution positions of the donor. Because of molecular building block shape control, linear molecular building blocks may form dense arrangements with strong intermolecular interactions, whereas Z-shaped molecular building blocks may form loose arrangements with weak intermolecular interactions, and quasi-Z-shaped molecular building blocks may form molecular arrangements with molecular interactions in a degree between linear and Z-shaped. Due to this molecular arrangement control, with a large difference in pore shape, the pore volume increased in Z-shaped>quasi-Z shaped>linear order. In addition, the three molecular building blocks self-assembled differently, resulting in different final microstructures: the Z-shaped block formed a rod-like structure, the quasi-Z-shaped block produced a mixture of small rods and granular forms, and the linear block led to a diamond-shaped morphology. As a result, the Z-shaped molecular building blocks exhibited the most sensitive vapor-induced fluorescence chromism of the three due to the loose molecular arrangement including the large V-shaped pores. Linear building blocks, on the other hand, exhibited selective vaporchromism to aromatic hydrocarbons due to a tight arrangement of molecules with small parallelogram-shaped pores.

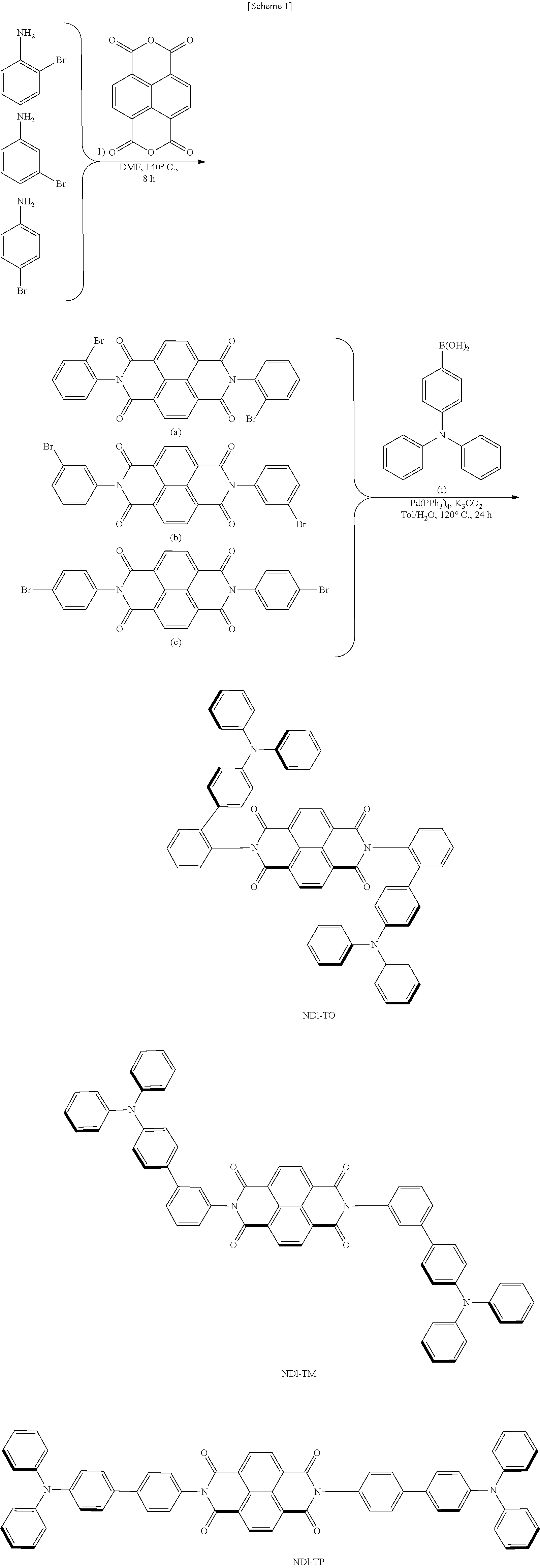

### Synthesis of Compound N,N′-bis(2-bromophenyl)-1,4,5,8-naphthalene Diimide (a)

Under an argon atmosphere, 1,4,5,8-naphthalene-tetracarboxylic dianhydride (0.5 g, 1.86 mmol) and 2-bromoaniline (4.28 mmol) were mixed in DMF (30 mL) and stirred for 8 hours at 150° C. After the reaction, it was cooled at room temperature and a precipitated solid mixture was filtered, thereby obtaining a product. The compound was obtained by purification through recrystallization using DMF. ¹H NMR (500 MHz, DMSO, ppm) δ8.80 (s, 4H), 7.89 (d, J=8.0 Hz, 2H), 7.71 (d, J=8.0 Hz, 2H) 7.64 (t, J=7.5 Hz, 2H), 7.51 (t, J=7.5 Hz, 2H).

### Synthesis of Compound N,N′-bis(3-bromophenyl)-1,4,5,8-naphthalene Diimide (b)

Under an argon atmosphere, 1,4,5,8-naphthalene-tetracarboxylic dianhydride (0.5 g, 1.86 mmol) and 3-bromoaniline (4.28 mmol) were mixed in DMF (30 mL) and stirred for 8 hours at 150° C. After the reaction, it was cooled at room temperature and a precipitated solid mixture was filtered, thereby obtaining a product. The compound was obtained by purification through recrystallization using DMF. ¹H NMR (500 MHz, DMSO, ppm) δ8.73 (s, 4H), 7.79 (s, 2H), 7.73 (d, J=7.5 Hz, 2H), 7.55 (t, J=8.0 Hz, 2H), 7.52 (d, J=8.0 Hz, 2H).

### Synthesis of Compound N,N′-bis (4-bromophenyl)-1,4,5,8-naphthalene Diimide (c)

Under an argon atmosphere, 1,4,5,8-naphthalene-tetracarboxylic dianhydride (0.5 g, 1.86 mmol) and 4-bromoaniline (4.28 mmol) were mixed in DMF (30 mL) and stirred for 8 hours at 150° C. After the reaction, it was cooled at room temperature and the precipitated solid mixture was filtered and collected. The compound was obtained by purification through recrystallization using DMF. ¹H NMR (500 MHz, DMSO, ppm) δ8.73 (s, 4H), 7.78 (d, J=9.0 Hz, 4H), 7.46 (d, J=8.5 Hz, 4H).

### Synthesis of Compound N,N′-bis(4-(triphenylamino)phenyl-2-yl)-1,4,5,8-naphthalene Diimide (NDI-TO)

Under an argon atmosphere, N,N′-bis(2-bromophenyl)-1,4,5,8-naphthalene diimide (0.3 g, 0.52 mmol), (4-(diphenylamino)phenyl)boronic acid (0.45 g, 2.61 mmol), Pd(PPh₃)₄ (10 mol %), and K₂CO₃ (0.65 g, 4.69 mmol) were mixed in Toluene/H₂O (v/v=40 mL/10 mL) and stirred for 24 hours at 110° C. After the reaction, it was cooled at room temperature, deionized water (50 mL) was added thereto, and then an organic layer was collected. A water layer was cleaned using methylene chloride (×3) to extract the remaining organic matter. After combining organic extracts, the organic layer was dried and filtered with anhydrous MgSO₄. The solvent was removed under decompression and the residue was purified with silica gel column chromatography using methylene chloride/hexane (V/V=1:1) as eluent to obtain purple powder. Yield: 0.17 g, 36%. ¹H NMR (500 MHz, CDCl₃, ppm) δ8.72 (s, 4H), 7.59-7.53 (m, 6H), 7.33 (d, J=7.5 Hz, 2H), 7.13 (d, J=9.0 Hz, 4H), 7.10 (t, J=7.5 Hz, 8H), 6.93 (t, J=7.5 Hz, 4H), 6.83 (d, J=8.5 Hz, 12H). ¹³C{¹H} (125 MHz, CDCl₃, ppm) δ162.82, 147.40, 147.14, 140.84, 132.75, 132.64, 131.13, 130.89, 129.66, 129.13, 129.06, 129.02, 128.46, 127.11, 126.76, 124.22, 123.15, 122.98. GC-MS (m/z) calcd. for C₆₂H₄₀N₄O₄: 904.30, Found: 904.4 [M]⁺. Anal. calcd. for C₆₂H₄₀N₄O₄: C, 82.28; H, 4.46; N, 6.19; O, 7.07. Found: C, 82.23; H, 4.43; N, 6.24; O, 7.10.

### Synthesis of Compound N,N′-bis(4-(triphenylamino)phenyl-3-yl)-1,4,5,8-naphthalene Diimide (NDI-TM)

Under an argon atmosphere, N,N′-bis(3-bromophenyl)-1,4,5,8-naphthalene diimide (0.3 g, 0.52 mmol), (4-(diphenylamino)phenyl)boronic acid (0.45 g, 2.61 mmol), Pd(PPh₃)₄ (10 mol %), and K₂CO₃ (0.65 g, 4.69 mmol) were mixed in Toluene/H₂O (v/v=40 mL/10 mL) and stirred for 24 hours at 110° C. After the reaction, it was cooled at room temperature, deionized water (50 mL) was added thereto, and then an organic layer was collected. A water layer was cleaned using methylene chloride (×3) to extract the remaining organic matter. After combining organic extracts, the organic layer was dried and filtered with anhydrous MgSO₄. The solvent was removed under decompression and the residue was purified with silica gel column chromatography using methylene chloride as eluent to obtain purple powder. Yield: 0.34 g, 72%. ¹H NMR (500 MHz, CDCl₃, ppm) δ8.87 (s, 4H), 7.73 (d, J=8.0 Hz, 2H), 7.64 (t, J=8.0 Hz, 2H), 7.53 (s, 2H), 7.50 (d, J=9.0 Hz, 4H), 7.30-7.25 (m, 10H), 7.13 (d, J=9.0 Hz, 12H), 7.04 (t, J=6.5 Hz, 4H). ¹³C{¹H} (125 MHz, CDCl₃, ppm) δ162.97, 147.71, 147.64, 142.45, 135.04, 133.84, 131.48, 129.89, 129.32, 127.96, 127.39, 127.29, 127.14, 126.85, 126.63, 124.57, 123.75, 123.10. GC-MS (m/z) calcd. for C₆₂H₄₀N₄O₄: 904.30, Found: 904.4 [M]⁺. Anal. calcd. for C₆₂H₄₀N₄O₄: C, 82.28; H, 4.46; N, 6.19; O, 7.07. Found: C, 82.24; H, 4.50; N, 6.22; O, 7.04.

### Synthesis of Compound N,N′-bis(4-(triphenylamino)phenyl-4-yl)-1,4,5,8-naphthalene Diimide (NDI-TP)

Under an argon atmosphere, N,N′-bis(4-bromophenyl)-1,4,5,8-naphthalene diimide (0.3 g, 0.52 mmol), (4-(diphenylamino)phenyl)boronic acid (0.45 g, 2.61 mmol), Pd(PPh₃)₄ (10 mol %), and K₂CO₃ (0.65 g, 4.69 mmol) were mixed in Toluene/H₂O (v/v=40 mL/10 mL) and stirred for 24 hours at 110° C. After the reaction, it was cooled at room temperature, deionized water (50 mL) was added thereto, and then an organic layer was collected. A water layer was cleaned using methylene chloride (×3) to extract the remaining organic matter. After combining organic extracts, the organic layer was dried and filtered with anhydrous MgSO₄. The solvent was removed under decompression and the residue was purified with silica gel column chromatography using methylene chloride as eluent to obtain purple powder. Yield: 0.24 g, 51%. ¹H NMR (500 MHz, CDCl₃, ppm) δ8.88 (s, 4H), 7.77 (d, J=8.5 Hz, 4H), 7.53 (d, J=9.0 Hz, 4H), 7.40 (d, J=8.5 Hz, 4H), 7.30 (t, J=7.0 Hz, 8H), 7.17 (d, J=8.5 Hz, 12H), 7.06 (t, J=7.0 Hz, 4H). ¹³C{¹H} (125 MHz, CDCl₃, ppm) δ163.03, 147.72, 147.63, 141.80, 133.91, 133.08, 131.50, 129.34, 128.72, 127.99, 127.79, 127.27, 127.11, 124.69, 123.54, 123.17. GC-MS (m/z) calcd. for C₆₂H₄₀N₄O₄: 904.30, Found: 904.4 [M]⁺. Anal. calcd. for C₆₂H₄₀N₄O₄: C, 82.28; H, 4.46; N, 6.19; O, 7.07. Found: C, 82.22; H, 4.49; N, 6.16; O, 7.13.

### Design Strategy of Single Molecule Building Block

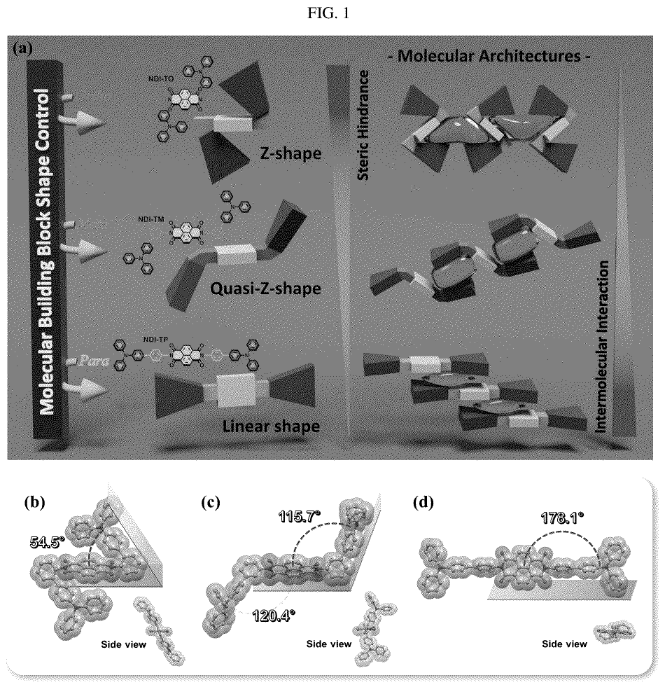

FIG. 1 is a view showing an example of a positional isomer dependent molecular building block (NDI-TO, NDI-TM, and NDI-TP) according to an embodiment of the disclosure. To build a self-assembled molecular building block that may efficiently control porous molecular arrangements, a positional isomer strategy based on the D-A-D system has been devised, as shown in FIG. 1(a), a schematic view of the design strategy. The building block is based on a D-A-D molecular skeleton consisting of an electron donor triphenylamine (TPA) and an electron acceptor NDI, and supports self-assembly through noncovalent interactions promoted by intermolecular donor-acceptor and hydrogen-binding sites. The NDI portion is a well-known electron deficiency class, widely used in vapochromic materials, and may provide four acyl groups as intermolecular hydrogen bond acceptors. In addition, the non-planar molecular structure of the TPA may cause porosity in the molecular arrangement. To provide the overall orientation of the molecular arrangement, phenyl groups were introduced between the TPA and NDI, resulting in changes in the shape of the molecular building blocks through ortho-, meta-, and para-substitution of the TPA. Thus, these molecular building blocks have the same chemical composition, but the single molecular structure is different, such as Z-shaped (NDI-TO), quasi-Z-shaped (NDI-TM), and linear (NDI-TP). FIG. 1(b) shows the crystal structure of NDI-TO, FIG. 1(c) shows the crystal structure of NDI-TM, and FIG. 1(d) shows the crystal structure of NDI-TP, respectively. Here, the C, N, and O atoms are indicated in gray, blue, and red, respectively. The H atom has been omitted for clarity.

A series of D-A-D system-based positional isomers of NDI-TO, NDI-TM and NDI-TP have been prepared in accordance with the synthesis procedures outlined in Scheme 1. The molecular structure of all products was determined by ¹H and ¹³C{¹H}-nuclear magnetic resonance, elemental analysis, mass spectrometry, and single-crystal structure analysis.

### Single Molecular Shape-Dependent Molecular Arrangement

Powder X-ray diffraction (PXRD) analysis was performed to characterize the original powder state of the positional isomer, showing that NDI-TO and NDI-TP have a microcrystalline structure while NDI-TM has an amorphous structure. In addition, NDI-TO and NDI-TP exhibited different PXRD patterns. Given that they have the same units, these differences in properties are due to different single-molecule forms caused by different donor substitution positions on the D-A-D molecular skeleton.

The shape of a single molecular structure based on a positional isomer and its packing arrangement has been identified by single crystal X-ray diffraction. The simulated pattern of single crystal structure of NDI-TO and NDI-TP is similar to the PXRD pattern of raw powder, which shows the similarity between the raw powder state structure and the corresponding single-crystal structure. As shown in FIGS. 1(b), 1(c), and 1(d), the angle between the donor and the acceptor increased in the order of NDI-TO(54.5°)<NDI-TM(115.7° and 120.4°)<NDI-TP(178.1°), which corresponds to a reduction in steric hindrance. These results indicate that “NDI-TO adopts a Z-shaped conformation, NDI-TM adopts a quasi-Z-shaped conformation, and NDI-TP adopts a linear shape, as expected. In addition, the crystalline structures of all compounds are consistent with the density functional theory (DFT)-optimized structure, indicating that the molecular structure has been reliably predicted by the DFT calculation. All compounds tended to form intermolecular hydrogen bonds due to the acyl of NDI, and different types of dimer/trimer were found for each crystal.

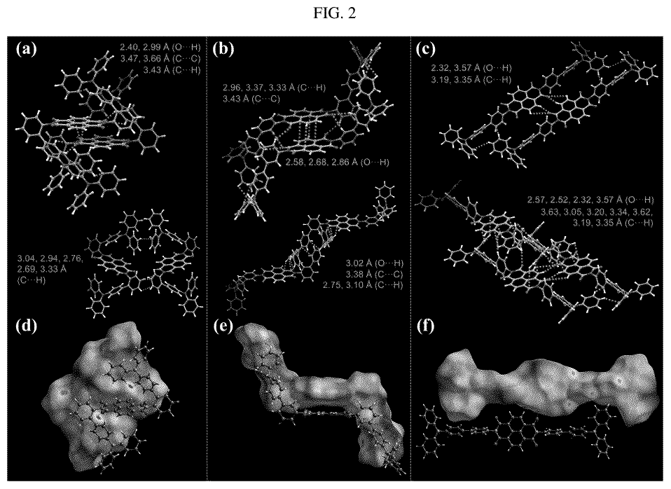

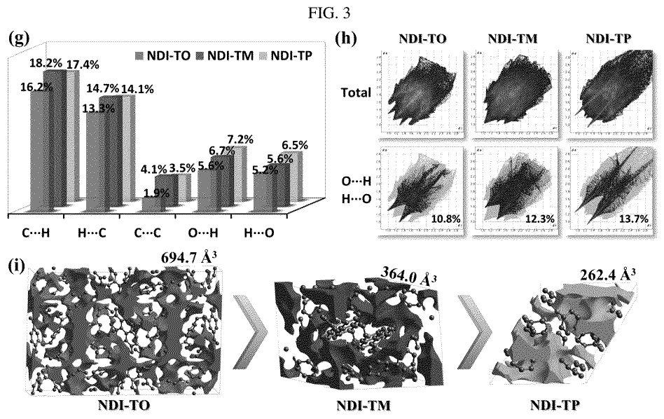

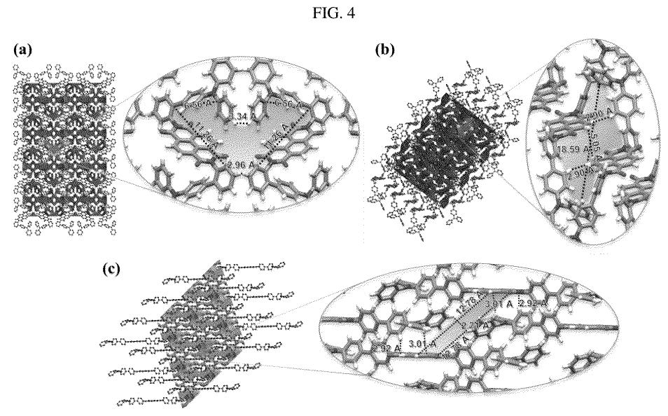

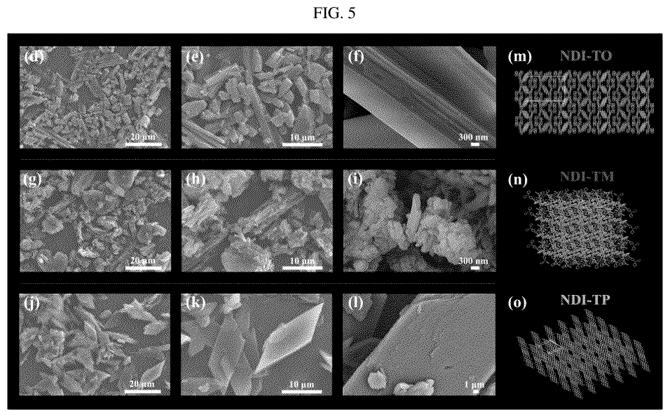

FIG. 2 is a view showing the stacking structures of (a) NDI-TO, (b) NDI-TM, and (c) NDI-TP, and the normalized distances (dnorm) mapped onto the Hirshfeld surfaces of (d) NDI-TO, (e) NDI-TM, and (f) NDI-TP according to an embodiment of the disclosure. In addition, FIG. 3 is a view showing (g) a bar chart of main non-covalent interactions % obtained from a two-dimensional (2D) fingerprint plot for all compounds, (h) a 2D fingerprint plot generated from dnorm for all interactions and O—H/H—O interactions, and (i) an example of void surfaces of NDI-TO, NDI-TM, and NDI-TP according to an embodiment of the disclosure. In addition, FIG. 4 is a view showing examples of a main void and void surfaces of four or more unit cells for (a) NDI-TO, (b) NDI-TM, and (c) NDI-TP according to an embodiment of the disclosure, and FIG. 5 is a view showing examples of low and high magnification Scanning Electron Microscopy (SEM) images for (d-f) NDI-TO, (g-i) NDI-TM and (j-l) NDI-TP, and examples of packing modes for (m) NDI-TO, (n) NDI-TM, and (o) NDI-TP according to an embodiment of the disclosure.

As shown in FIG. 2(a), the Z-shaped NDI-TO adopts stepwise packing and antiparallel packing during synthesis due to steric hindrance. In essence, it does not allow intermolecular donor-acceptor interactions, resulting in intermolecular space formation and insufficient intermolecular interactions. Unlike NDI-TO, NDI-TM adopts a quasi-Z-shaped conformation with a long intramolecular distance between TPA and NDI. As shown in FIG. 2(b), NDI-NDI and TPA-TPA superposition configurations are thus possible, resulting in various molecular interactions such as x-x, donor-donor, donor-acceptor, and hydrogen bonds. In addition, unlike the two cases of the Z-shaped series above, in the case of linear NDI-TP, strong hydrogen-binding interactions (2.32˜3.57 Å of O—H—C) as well as intermolecular donor-accepter interactions are observed as shown in FIG. 2(c).

Hirshfeld surface analysis is useful for quantifying and evaluating non-covalent interactions with molecular packing of positional isomers. In FIG. 2(d-f), the red areas on the resulting Hirshfeld surfaces, which indicate contacts shorter than the van der Waals limit, shows differences in key intermolecular interactions depending on positional isomerism. The Hirshfeld surface of NDI-TO indicates that the O—H—C hydrogen bond between molecules and C—H of the phenylinker-phenylinker contact exist as key intermolecular interactions. In contrast, the red dot is scattered on the Hirshfeld surface of NDI-TM, indicating that various intermolecular interactions (hydrogen bonds and NDI-NDI, TPA-TPA and NDI-TPA contacts) contribute to the stabilization of the packing structure of NDI-TM. For linear NDI-TP, the dark red dots on the sides of NDI and TPA indicate that the key intermolecular interactions are hydrogen bonds formed by lateral and parallel contact between adjacent molecules.

These intermolecular interactions were compared using the 2D fingerprint plot in FIG. 2(h), and the percentage contributions of the major interactions are presented as a bar chart in FIG. 2(g). Compared with NDI-TM and NDI-TP, the contribution of C—H/H—C, and C—C contacts associated with intermolecular donor-acceptor and same-unit interactions, was the smallest in NDI-TO. In the case of NDI-TM, the contribution of C—H/H—C and C—C contacts slightly exceeded the contribution of NDI-TP, but this was primarily attributed to intermolecular same-unit contact interactions. In particular, the contribution of O—H/H—O contacts corresponding to intermolecular hydrogen bonds increased in the order of NDI-TO<NDI-TM<NDI-TP. In addition, in the lower left corner of the fingerprint plot, the spike of NDI-TP is larger than the spike of NDI-TO and NDI-TM, indicating that the intermolecular hydrogen bond of NDI-TP is stronger than the spike of the Z-shaped series. Therefore, linear NDI-TP adopts the most dense packing mode with strong intermolecular interaction, whereas Z-shaped NDI-TO adopts the most loose packing mode with weak intermolecular interaction. Therefore, FIGS. 2(i) and 3(a-c), the volume of voids increases in the order of NDI-TP (262.4 Å)<NDI-TM (364.0 Å)<NDI-to (694.7 Å), especially there are definite differences in the shape and size of the main pores between positional isomers. The Z-series building block NDI-TO has a relatively large V-shaped main pore formed by loose antiparallel packing, while the linear building block NDI-TP has a relatively small parallelogram-shaped main pore due to its rich intermolecular interaction arrangement. The quasi-Z-shaped building block NDI-TM represents a moderately sized zigzag-shaped main pore due to the degree of intermolecular stacking between NDI-TP and NDI-TO. According to these results, the pore of the D-A-D system-based positional isomer formed by this arrangement may function as a guest molecular recognition site in the stimulus reactant, and the pore properties controlled by the shape of the single molecular building block are expected to cause characteristic vapofluorochromism/vapochromism.

The shape and microstructure of the positional isomers were then examined by scanning electron microscopy (SEM). The observed positional isomers showed different microscopic forms. As shown in FIG. 5(d-f), the NDI-TO may self-assemble into micro-sized bars. Such homogeneity in shape indicates that, the structural properties of the Z-type building block forces the intermolecular interactions to follow a limited path arrangement due to an insufficient and limited number of molecular packing modes, resulting in the final shape. However, as shown in FIG. 5(g-i), with the presence of various packing modes due to the quasi-Z-shaped building block, the self-assembly of NDI-TM formed a collection of granules derived from the compressed bar and the reduced bar. As shown in FIG. 5(j-l), unlike the Z-shaped series, the linear NDI-TP may self-assemble into a hierarchical rhombic structure. This indicates that the molecular packing mode, which is composed of many strong intermolecular interactions due to the linear building block, is the driving force behind the formation of the rhombus, the final form of NDI-TP. The form of the assembly is characterized via SEM and is similar to the unit cell of the crystal structure, as shown in FIG. 5(m-o), indicating that molecular and microscale arrangements are closely related. Thus, manipulating the shape of the single molecular building block through the positional isomer, may adjust the molecular arrangement and porosity and affect the final microstructure form.

### Photophysical Properties

The steady state absorption and emission spectra of positional isomers were measured in dichloromethane (DCM) solutions. In the absorption spectra, all compounds have represented an absorption band with a wide absorption band at ˜308 nm and another band with three vibronic features at 320-380 nm, which may be attributed to locally excited transitions of TPA and NDI, respectively. In particular, the absorption spectra of all compounds exhibited almost identical absorption initiation at about 393 nm, and the experimental and calculated band-gap energies were similar. Similar to the ground state, the emission spectra of all compounds represents a vibronic structured emission band at 400-430 nm and a structureless broad emission band at ˜453 nm. In addition, the positional isomer exhibited the same monoexponential fluorescence decay with a similar fluorescence quantum yield of about 0.05 and a similar lifetime value of 3.7 ns. These results suggest that due to the weakened positional isomeric junction effect induced by N-imide substitution on the NDI core, all compounds exhibit a similar emission origin, and their absorption spectra show weak dependence on solvent polarity. In fact, time-Dependent Density Functional Theory (TD-DFT) calculations support less sensitive ground conditions by showing that the lowest energy singlet transition has the characteristics of Intramolecular Charge Transfer (ICT) but has a lower oscillator strength. In contrast, the emission spectra of all compounds showed redshift as solvent polarity increased, indicating that the emission state originated from an ICT state sensitive to the surrounding environment. In particular, all compounds have similar solvatochromic shifts (˜68 nm) and the Lippert-Mataga plot slopes, indicating that ICT characteristics are similar in excited state. Thus, due to weakening of the positional isomer effect by N-imide positional substitution, the optical properties of all compounds were similar in solution states.

In the solid state, the emission spectra of all compounds exhibited fluorescence quenching. Aggregation-induced emission spectra measured in tetrahydrofuran (THF)/water mixtures with different water volume ratios exhibited the same trend. These results indicate that in the solid state the compound has a weak fluorescent background signal for application as VOC-sensing optical sensor materials.

### Vapochromic Behavior

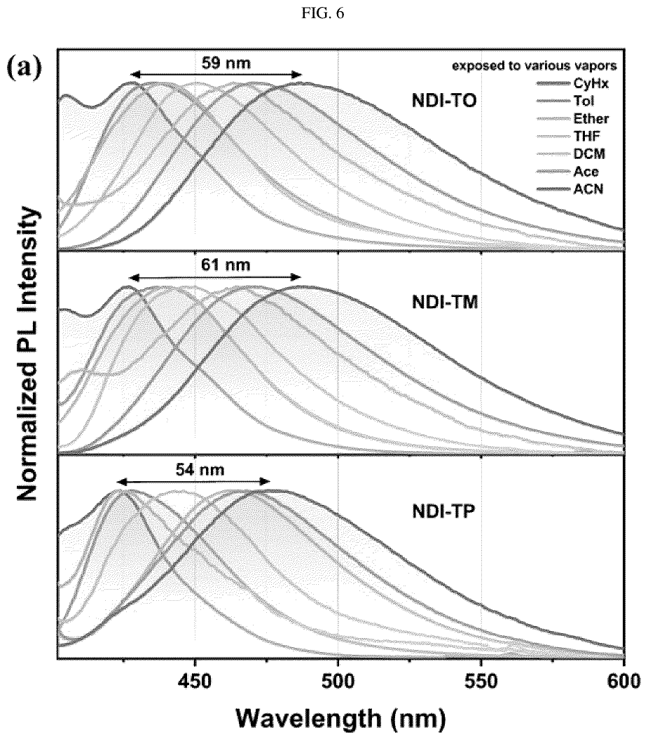

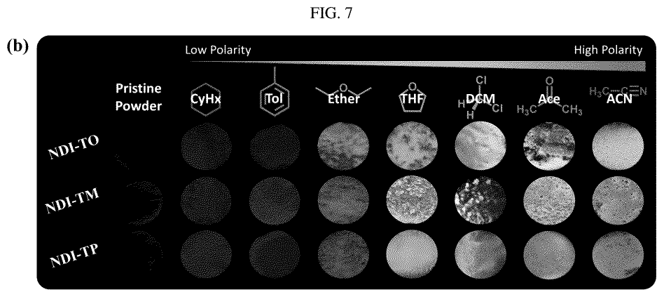

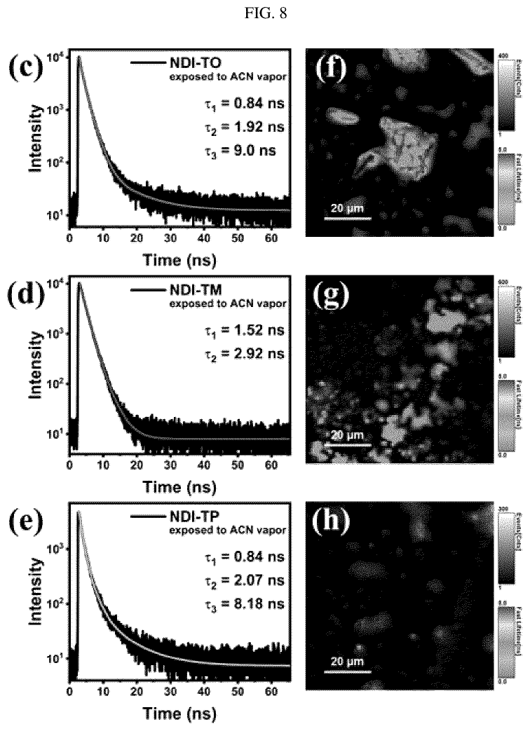

FIG. 6 shows examples of emission spectra of NDI-TO, NDI-TM, and NDI-TP when exposed to various vapors according to an embodiment of the disclosure. In addition, FIG. 7 is a view showing examples of images of NDI-TO (top), NDI-TM (middle), and NDI-TP (bottom) powder in its original state exposed to various vapors under a 365 nm UV lamp according to an embodiment of the disclosure. In addition, FIG. 8 is a view examples of time-resolved photoluminescent decays of (c) NDI-TO, (d) NDI-TM and (e) NDI-TP extracted from the relevant fluorescence lifetime images, and examples of fluorescence lifetime images of (f) NDI-TO, (g) NDI-TM and (h) NDI-TP when in contact with ACN vapor according to an embodiment of the disclosure.

Solid samples prepared from sealed fluorescent cells were exposed to guest vapors and then emission spectra were collected for each system to investigate the properties of vapor-induced fluorescence chromism of isomer compounds. Unlike the initial state, a broad emission band of ˜426 nm was observed in the emission spectra of all compounds exposed to cyclohexane (CyHx) vapor. After that, when CyHx vapor was removed from the air at room temperature, all compounds returned to their initial emission-off state. This fluorescence on-off transition of the compound indicates that the transition between two different states can be reversed several times without fatigue response due to repeated exposure and removal of CyHx vapor. In particular, the turned-on vapor-induced fluorescence chromism reacted to a variety of emission colors under various organic vapor stimuli, and the emission spectra of all compounds were redshifted. Redshift increased in the order of CyHx<toluene (Tol)<ethyl ether (Ether)<THF<DCM<acetone (Ace)<acetonitrile (ACN) vapor, depending on solvent polarity. As a proof of concept, a polarity-sensitive turned-on vapor-induced fluorescence chromism was demonstrated using NDI-TM as an example. NDI-TM filled in the NDI letter mold under ultraviolet (UV) radiation showed no emission. However, contact with ACN vapor produced a light green emission. Upon removal of ACN vapor, the compound returned to its non-emissive state, and then when exposed to CyHx vapor, it produced a blue emission. To determine the cause of this vapor-induced fluorescence chromism, lifetime imaging was performed using a time-resolved fluorescence microscope compared to a single molecular emission spectrum. The vapor-induced fluorescence chromism emission spectrum, which exhibited redshift as polarity increases, was similar to the corresponding emission wavelength region observed the solution, but was accompanied by the disappearance of most vibronic structures and, in some cases, the emergence of an excimer emission band. In particular, as shown in FIG. 8(c-h), the decay profile of the positional isomers observed in the fluorescence lifetime image when in contact with ACN vapor showed distinct life spans with two or three exponential components, as opposed to those observed in the solution. According to these results, D-A-D positional isomers, which exhibit emission quenching characteristics in its pristine state, activate Charge Transfer (CT)-based emission upon vapor stimulation, and this CT-based vapor-induced fluorescence chromism emission occurs not in the simple release state of the solution, but in the complex excitation state of various pathways.

Interestingly, although the positional isomers exhibit similar ICT characteristics in solution, they displays distinct vapor-induced fluorescence chromic shifts in the solid state. As shown in FIGS. 6 and 7, the vapor-induced fluorescence chromism shift of Z-series NDI-TO (59 nm) and NDI-TM (61 nm) was greater than that of linear NDI-TP (54 nm), and the emission spectrum changes in non-polar environments such as CyHx, Tol and Ether were relatively clear. In particular, although o-, m-, and p-xylene (ortho-, meta-, para-xylene) isomers are challenging to differentiate due to their similar physicochemical properties, when NDI-TO (11 nm) contacts vapors thereof, it demonstrated a significantly greater vapor-induced fluorescence chromism shift in emission maxima among the xylene isomers, compared to NDI-TM (2 nm) and NDI-TP (5 nm), and the release of the redshift was different from the solution state. These results indicate that the Z-shaped NDI-TO has vapor-induced fluorescence chromism most sensitive to polar environments among the D-A-D system-based positional isomers. In other words, the Z-shaped building block NDI-TO is advantageous in converting to a CT-based emission state upon vapor contact. In NDI-TO, the formation of loose molecular arrangements with large V-shaped pores can facilitate the penetration of guest molecules and induce active interactions with them. In contrast, linear building block NDI-TP exhibits vapor-induced fluorescence chromism, which is less sensitive to polar environments because of the difficulty of guest penetration and interaction with the guest due to the formation of a close molecular arrangement with relatively small parallelogram pores. The quasi-Z-shaped NDI-TM, which forms a moderately sized zigzag-shaped main pore, exhibited vapor-induced fluorescence chromism in a degree between that of NDI-TO and NDI-TP. Thus, the CT-based turn-on vapor-induced fluorescence chromic behavior was determined by a single molecular shape-dependent molecular arrangement.

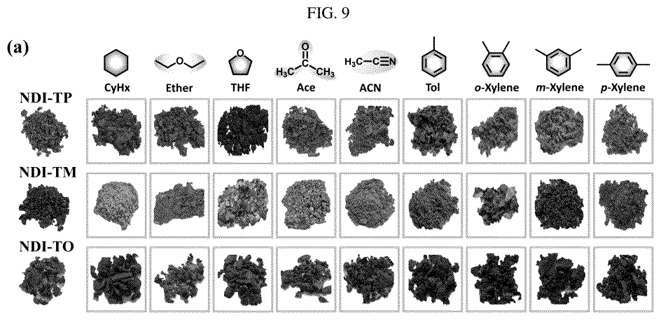

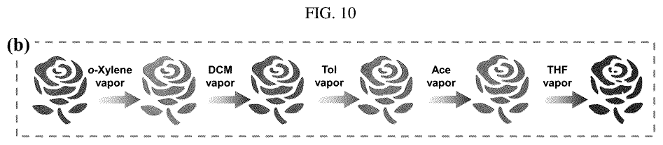

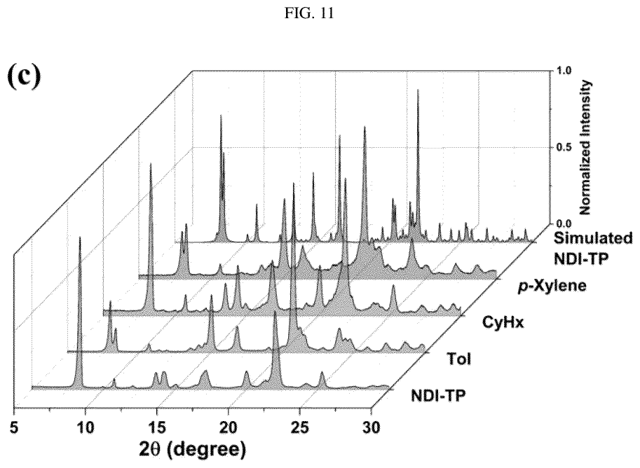

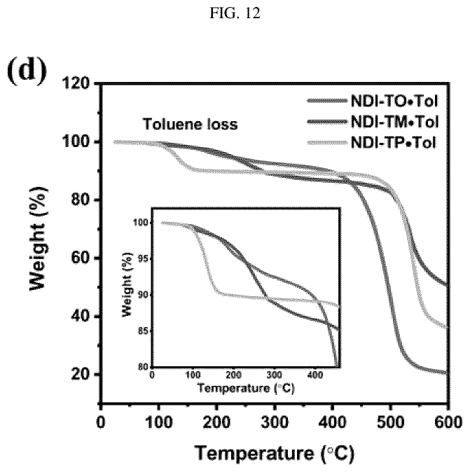

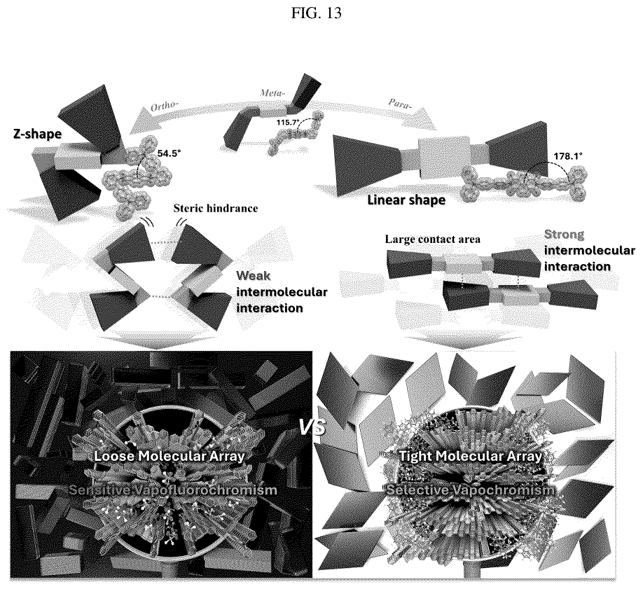

FIG. 9 is a view showing examples of images of NDI-TP (top), NDI-TM (middle), and NDI-TO (bottom) powder after exposure to various vapors under ambient light for 24 hours according to an embodiment of the disclosure. FIG. 10 is a view showing an example of changes in color of a rose drawn with NDI-TP using a mask on filter paper under ambient light when sequentially exposed to o-xylene, DCM, Tol, Ace, and THE vapors for 24 hours according to an embodiment of the disclosure. FIG. 11 is a view showing an example of an original state of NDI-TP and experimental PXRD patterns of NDI-TP powder after exposure to Tol, CyHx and p-xylene vapors, along with a simulation pattern of the single crystal structure of NDI-TP according to an embodiment of the disclosure. In addition, FIG. 12 is a view showing examples of TGA curves of NDI-TO, NDI-TM, and NDI-TP powder samples after exposure to Tol vapor according to an embodiment of the disclosure. In addition, FIG. 13 is a view showing an example of the correlation between a single molecular shape-dependent molecular arrangement and vapochromism behavior according to an embodiment of the disclosure.

Vapochromism was tested by exposing positional isomer powders to each of the aforementioned saturated organic vapors for 24 hours at room temperature. As shown in FIG. 9, the Z-shaped NDI-TO did not exhibit significant color changes when observed with the naked eye, while the quasi-Z-shaped NDI-TM showed variable and irregular vapor chromic behavior. Interestingly, linear NDI-TP exhibits selective vapor chromism, displaying a purple color for small aromatic hydrocarbons such as Tol and xylene isomers, and a dark blue or purple color for other organic compounds. As shown in FIG. 10, as a proof-of-concept experiment, a color-changing rose was demonstrated by utilizing the vapor chromic properties of the linear NDI-TP, which selectively responds to aromatic hydrocarbons. When sequentially exposed to o-xylene, DCM, Tol, Ace, and THE vapors, the purple rose drawn with NDI-TP using a rose-shaped mask on a filter paper showed a purple color change only to aromatic hydrocarbon vapors. In particular, even when applied in powder form or coated onto filter paper via drop-casting, NDI-TP exhibited selective vapor chromic behavior toward aromatic hydrocarbons. In order to more specifically compare this vapor chromic behavior, PXRD patterns of samples exposed to CyHx, Tol and p-xylene vapors were identified and compared to their original state, as shown in FIG. 11. As expected, all NDI-TO samples exhibited a PXRD pattern similar to the original powder, indicating that there was no significant change in the molecular arrangement of NDI-TO despite vapor exposure. For NDI-TM, the PXRD pattern was all different, with wide peaks indicating amorphous when exposed to CyHx and sharp peaks indicating microcrystalline when exposed to Tol and p-xylene. These results mean that NDI-TM exists in various forms of molecular arrangement, including amorphous structures, depending on the surrounding environment. Unlike the Z-type series, the PXRD pattern of NDI-TP exposed to CyHx was similar to that of its pristine state, and the pattern of NDI-TP exposed to Tol and p-xylene was also similar. Consistent with visual observation, these results may indicate that linear NDI-TP exhibits different selective vapor chromic behaviors toward aromatic and non-aromatic hydrocarbons. In addition, as shown in FIG. 12, a thermogravimetric analysis (TGA) was performed on the powder of each positional isomer exposed to Tol to investigate the presence of vapor molecules. Overall, NDI-TO, NDI-TM and NDI-TP decomposed at 458.6, 508.6 and 512.6° C. respectively, showing high thermal stability. The TGA curves of NDI-TO and NDI-TM between 25 and 300 or 400° C. exhibited multiple decomposition steps, while NDI-TP exhibited one decomposition step, wherein weight losses of 7.5%, 13.4%, and 10.5% were observed. These weight losses are due to the loss of the Tol molecule, and the molar ratio (Tol/each compound) was calculated at 0.8:1 (NDI-TO), 1.5:1 (NDI-TM), and 1.2:1 (NDI-TP). According to these results, the Z-shaped building block NDI-TO exhibited inert vapochromic behavior due to its loose molecular packing with large V-shaped voids, which minimizes changes in molecular arrangement even in the presence of guest molecules. Furthermore, the quasi-Z-shaped building block NDI-TM has multiple permissible intermolecular stacking modes, which may lead to irregular vapochromic behavior. Although the tight molecular packing of the linear building block NDI-TP, with small parallelogram-shaped voids, is not ideal for vapochromic fluorescence, it exhibits selective vapochromic behavior because relatively large structural perturbations can be induced in the molecular arrangement by aromatic hydrocarbons occupying the intermolecular spaces. Thus, using positional isomers to manipulate the shape of D-A-D based molecular building blocks has proven to be a promising option for controlling molecular arrangements, which ultimately leads to changes in vapor-induced fluorescence chromism and vapochromic behavior, as shown in FIG. 13. The correlation between single-molecular-shape dependent molecular arrangements and vapochromic behavior may be used to develop efficient strategies for designing new vapor-induced fluorescence chromism and vapochromic materials.

As such, according to embodiments of the disclosure, the donor-acceptor-donor positional isomer can be utilized to manipulate the monomolecular structure to provide an organic material in which an organic porous molecular arrangement is controlled. In addition, it is possible to provide vapochromic organic materials that are inexpensive, easy to synthesize, and suitable for mass production due to the absence of rare metals. In addition, organic porous molecular arrangements constructed via non-covalent interactions possess more flexible structures than conventional MOFs and COFs, allowing significant structural deformation in response to stimuli such as guest molecules, thereby enabling the development of high-performance vapochromic organic materials. In addition, by controlling the shape of the positional isomer, organic porous molecular arrangements can be readily tuned, enabling selective modulation of vapochromic performance and properties based on the arrangement, thereby offering advanced vapochromic organic materials.

The above explanation is only an exemplary explanation of the technical thought of the disclosure, and a person with ordinary knowledge in the technical field to which the disclosure belongs will be able to make various modifications and variations to the extent that it does not deviate from the essential characteristics of the disclosure. Therefore, the embodiments described in the disclosure are intended to explain, not limit, the technical ideas of the disclosure, and should not be construed as being restricted thereto. The scope of protection of the disclosure shall be interpreted by the claims below, and all technical ideas within the equivalent scope shall be interpreted as being included in the scope of rights of the disclosure.

1. A Donor-Acceptor-Donor (D-A-D) positional isomer-based organic material compound containing Naphthalene diimide (NDI).

2. The organic material compound according to claim 1, wherein the donor-acceptor-donor positional isomer is utilized to manipulate a single molecular structure to control an organic porous molecular arrangement.

3. The organic material compound according to claim 2, wherein vaporchromic properties for Volatile Organic Compounds (VOCs) is selectively induced by controlling the organic porous molecular arrangement.

4. The organic material compound according to claim 1, wherein at least one of color and luminescence color of the organic material compound is changed in response to a particular gas or vapor.

5. The organic material compound according to claim 1, wherein the organic material compound is expressed by the following formula 1.

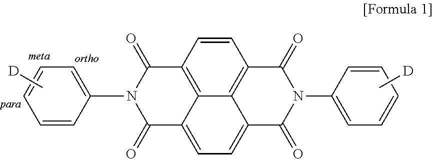

6. The organic material compound according to claim 1, wherein a phenyl group is introduced between the napthalendiamide and a donor unit, and the donor unit in the donor-acceptor-donor positional isomer is substituted at one of positions of ortho-, meta-, and para-of the phenyl.

7. The organic material compound according to claim 6, wherein the shape of a molecular building block of the donor-acceptor-donor positional isomer is controlled to be one of Z-shaped, quasi-Z-shaped, or linear as the donor unit is substituted at one of the positions of ortho-, meta-, and para- of the phenyl.

8. The organic material compound according to claim 7, wherein a pore volume decreases in the order of Z-shaped, quasi-Z-shaped, and linear according to the shape of the molecular building block.

9. The organic material compound according to claim 7, wherein the donor unit in the donor-acceptor-donor positional isomer includes triphenylamine (TPA).

10. The organic material compound according to claim 1, wherein a donor unit in the donor-acceptor-donor positional isomer is expressed by the following formula 2.

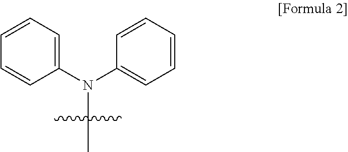 [SMILES: *N(c1ccccc1)c1ccccc1]

11. The organic material compound according to claim 1, wherein a donor unit in the donor-acceptor-donor positional isomer is expressed by the following formula 3.

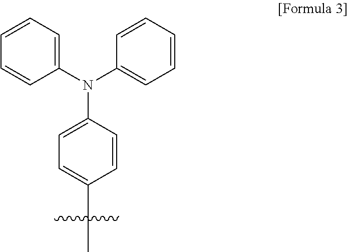 [SMILES: *c1ccc(N(c2ccccc2)c2ccccc2)cc1]

12. The organic material compound according to claim 1, wherein a donor unit in the donor-acceptor-donor positional isomer is expressed by the following formula 4.

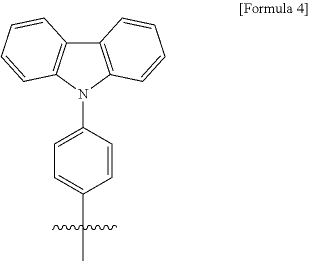 [SMILES: *c1ccc(-n2c3ccccc3c3ccccc32)cc1]

13. The organic material compound according to claim 1, wherein a donor unit in the donor-acceptor-donor positional isomer is expressed by the following formula 5.

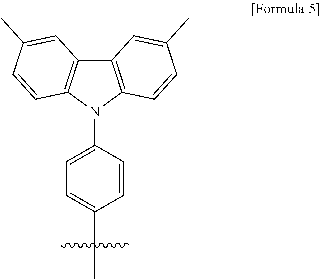

14. The organic material compound according to claim 1. wherein a donor unit in the donor-acceptor-donor positional isomer is expressed by the following formula 6.

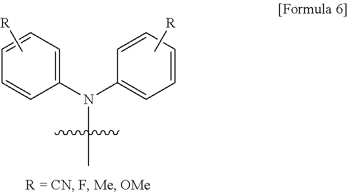

15. The organic material compound according to claim 1, wherein a donor unit in the donor-acceptor-donor positional isomer is expressed by the following formula 7.

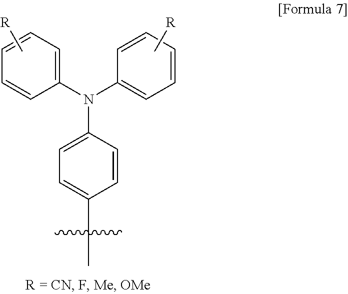

16. The organic material compound according to claim 1, wherein a donor unit in the donor-acceptor-donor positional isomer is expressed by the following formula 8.

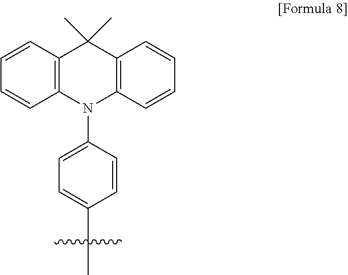 [SMILES: *c1ccc(N2c3ccccc3C(C)(C)c3ccccc32)cc1]

17. The organic material compound according to claim 1, wherein a donor unit in the donor-acceptor-donor positional isomer is expressed by the following formula 9.

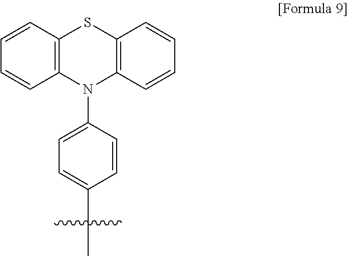 [SMILES: *c1ccc(N2c3ccccc3Sc3ccccc32)cc1]

18. The organic material compound according to claim 1. wherein a donor unit in the donor-acceptor-donor positional isomer is expressed by the following formula 10. 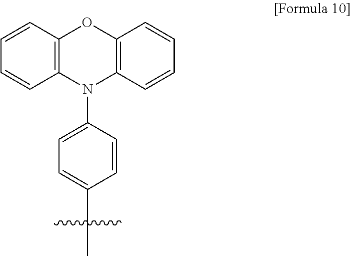 [SMILES: *c1ccc(N2c3ccccc3Oc3ccccc32)cc1]
